(19) United States
(12) Patent Application Publication
(10) Pub. No.: US 2005/0130184 A1
(43) Pub. Date: Jun. 16, 2005

(54) Enhanced promoters for synthesis of small hairpin RNA

(75) Inventors: Zuoshang Xu, North Grafton, MA (US); Xugang Xia, Westborough, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA (US)

(21) Appl. No.: 10/894,721

(22) Filed: Jul. 19, 2004

Related U.S. Application Data
(60) Provisional application No. 60/488,312, filed on Jul. 18, 2003.

(51) Int. Cl. C12Q 1/68; G06F 19/00; G01N 33/48; G01N 33/50; C07H 21/02; C12N 9/22

(52) U.S. Cl. 435/6; 435/69.1; 435/199; 435/320.1; 435/325; 536/23.1; 702/20

(57) ABSTRACT

The present invention provides compositions for RNA interference and methods of use thereof. In particular, the invention provides small hairpin RNAs (shRNAs) having modified promoters, including the Pol III U6 promoter, which may be used to increase the potency of shRNA by increasing the expression level. Modifications include constructs with a Pol II enhancer, such as the cytomegalovirus (CMV) enhancer, immediate-early promoter near the Pol III, e.g., U6 promoter, either upstream or downstream from the shRNA sequence and in either forward or backward orientation. Such constructs are useful for increasing the expression of the shRNA, thereby enhancing inhibition of a single nucleotide mismatched mutant allele. Functional and genomic and proteomic methods are featured. Therapeutic methods are also featured.

### RELATED APPLICATIONS

This patent application claims the benefit of U.S. Provisional Patent Application Ser. No. 60/488,312, entitled “Enhanced Promoters for Synthesis of Small Hairpin RNA”, filed Jul. 18, 2003. The entire contents of the above-referenced provisional patent application are incorporated herein by this reference.

### BACKGROUND OF THE INVENTION

Mutations in Cu, Zn superoxide dismutase (SOD1) gene cause a subset of amyotrophic lateral sclerosis, a neurodegenerative disease that leads to motor neuron degeneration, paralysis and death (Brown and Robberecht, 2001; Siddique and Lalani, 2002). It has been well established that mutant SOD1 causes motor neuron degeneration by acquisition of a toxic property (Cleveland and Rothstein, 2001). However, neither the molecular basis of this toxic property nor mechanism that leads to motor neuron death is understood. Because of this incomplete understanding of the disease mechanism, rational design of therapy has not produced robust efficacious outcomes. On the other hand, because the toxicity that kills motor neurons originates from the mutated protein (Cleveland and Rothstein, 2001), decrease of the mutant protein should alleviate or even prevent the disease. This may be achieved by RNA interference (RNAi).

RNA interference (RNAi) can mediate sequence-selective suppression of gene expression in a wide variety of eukaryotes by introducing short RNA duplexes (small interfering RNAs or siRNAs) with sequence homologies to the target gene (1,2). Furthermore, small hairpin RNAs (shRNAs) transcribed in vivo under the control of RNA polymerase III (Pol III) promoters can trigger degradation of corresponding mRNAs similar to siRNAs and inhibit specific gene expression (3-11). Constructs that synthesize shRNA have been incorporated into viral vectors and these vectors can mediate RNAi in culture as well as in vivo (12-16). Therefore, Pol III-shRNA constructs may be developed to mediate long term silencing of dominant, gain-of-function type of mutant genes that cause diseases.

Diseases caused by dominant, gain-of-function mutations develop in people bearing one mutant and one wild-type copy of the gene. Some of the best-known examples of this class are neurodegenerative diseases, including Huntington's, a subset of amyotrophic lateral sclerosis (ALS) and rare, familial forms of the otherwise common Alzheimer's and Parkinson's diseases (Taylor et al., 2002). In all these diseases, the exact pathways whereby the mutant proteins cause cell degeneration are not clear, but the origin of the cellular toxicity is known to be the mutant protein. Thus, selectively lowering or eliminating the mutant protein is a key step in developing effective therapies. Until recently, it was not clear how specific down-regulation of a wide variety of mutant proteins could be achieved. Although opinions vary (17-19), many experiments have shown that siRNAs and shRNAs can discriminate between mRNAs that differ at a single nucleotide and selectively degrade the perfectly matched mRNA, while leaving the mRNA with a single nucleotide mismatch unaffected (7,9,12,17,20). The discriminating siRNAs or shRNAs must include the altered nucleotide in their sequences, and in most instances, the optimal design places the altered nucleotide near or at the middle of the siRNA or shRNA. This limits the sequence selection in designing siRNA or shRNA around the site of mutation. Because the sequence of siRNA or shRNA greatly influences the efficacy of RNAi (18,21), the sequences surrounding the mutation site may not be optimal and could produce poor inhibitors of the mutant gene. Accordingly, the present invention provides compositions and methods for overcoming this limitation by, for example, increasing the dose of siRNA and thereby enhancing the expression of shRNA.

### SUMMARY OF THE INVENTION

The present invention is based on the discovery that small hairpin RNAs (shRNAs) transcribed by RNA polymerase III (Pol III) promoters can trigger sequence-selective gene silencing in culture and in vivo, and therefore, may be developed to treat diseases caused by, for example, (i) aberrant modification or mutation of a gene encoding a protein; (ii) mis-regulation of a gene; and (iii) aberrant post-translational modification of a protein. In addition, the compositions and methods described herein may be useful in the treatment of disease caused by aberrant expression, e.g., overexpression, of a gene. Thus, the methods and compositions of the invention may be used to treat diseases that develop in people bearing one mutant and one wild-type gene allele, e.g., dominant, gain-of-function gene mutations. While the mutant is toxic, the wild type performs important functions. Thus, the ideal therapy must selectively silence the mutant but maintain the wild type expression.

Accordingly, the present invention features modified promoters, for example, Pol III promoters (e.g., the U6 promoter), which can be used to increase the potency of shRNA-mediated RNA interference (RNAi) by increasing the expression level of a shRNA. In particular, the invention features constructs, which are modified by placing Pol II enhancer sequences, for example, cytomegalovirus (CMV) enhancer sequences, near the Pol III promoter (e.g., the U6 promoter), either upstream or downstream from the shRNA sequence and in either forward or backward orientation. The data exemplified herein demonstrates that Pol II enhancer (e.g., the CMV enhancer) can enhance Pol III promoter (e.g., U6 promoter) activity and increase the production of shRNA. Experimental analysis has confirmed that the addition of the Pol II enhancer, (e.g., CMV enhancer), in all four configurations increased the expression of the shRNA. The constructs described herein have tremendous utility not only as research tools, for example, as selective inhibition of gene expression (e.g., mutant gene expression) in vitro and in vivo, but also are useful for developing therapeutic agents for treating diseases.

Preferred constructs of the invention encode a shRNA capable of selectively silencing a mutant allele encoding Cu, Zn superoxide dismutase (SOD1^{G93A}) allele that causes amyotrophic lateral sclerosis. Based on these discoveries, it was found that increased expression lead to enhanced inhibition of the single nucleotide mismatched mutant allele. Thus, this enhanced Pol III promoter (e.g., the U6 promoter) is useful where limited choices of shRNA sequences preclude the selection of highly efficient RNAi target region.

Accordingly, the present invention features a construct comprising a nucleotide sequence encoding a shRNA operably linked to a Pol III promoter, (e.g., a U6 promoter, a H1 promoter, or a tRNA promoter), and a Pol II enhancer (e.g., a CMV enhancer). In another embodiment, the invention provides a construct comprising a nucleotide sequence encoding a shRNA operably linked to a Pol II promoter, e.g., a CMV promoter, and a Pol III enhancer. The invention provides nucleic acid sequences set forth as SEQ ID NOS:1-7.

In one embodiment, the constructs of the invention include a shRNA sequence sufficiently complementary to a target mRNA to mediate degradation of said target. The target mRNA may encode a wild type protein or a mutant protein, e.g., disease-causing mutant, such as a gain-of-function mutant, SOD1, SOD1^{G93A}, and SOD1^{G85R}. The mutant protein may be causative of a disease or disorder, including neurological and neurodegenerative diseases or disorders. Neurodegenerative diseases and disorders may include, but are not limited to, Lou Gehrig's disease, amyotrophic lateral sclerosis (ALS), Alzheimer's disease, Parkinson's disease, Adrenoleukodystrophy (ALD), and dementia.

The invention also features a cell, including an animal cell, or a vector, e.g., a viral, AAV, Lentiviral, Adenoviral or Herpes vector, that contains a construct of the present invention. In addition, the invention provides a cell containing a vector of the present invention.

A construct of the present invention may contain an enhancer that is upstream or downstream from the promoter. In another aspect of the invention, a construct featured herein may contain an enhancer in a forward or backward orientation. The constructs of the invention may further include a pharmaceutically acceptable carrier.

In one embodiment, the invention provides a nonhuman transgenic animal carrying a transgene that contains a construct of the invention. Additionally, a nonhuman homologous recombinant animal which contains a cell of the invention is provided.

The invention features a method of introducing, e.g., transfecting, into a cell a construct of the invention under conditions such that shRNA expression is increased, thereby enhancing RNAi. In one aspect of the invention, the cell may be present in a subject or a cultured cell. In another aspect of the invention, the introduction of the construct may include infecting the cell with a viral vector.

The invention provides a method for enhancing RNAi in a subject by administering a construct or composition of the invention, thereby enhancing RNAi in a subject. The invention also features a method for selectively inhibiting mutant gene expression in vivo or in vitro by introducing into a host cell a construct of the invention under conditions such that shRNA is expressed, thereby inhibiting mutant gene expression. In one aspect of the invention, the shRNA does not inhibit expression of the wild type allele.

The present invention also features a method for treating a disease, including a neurodegenerative disease, in a subject by administering a construct or composition of the invention, thereby treating a disease in a subject. In one aspect of the invention, the disease may be caused by a mutation that is a dominant, gain-of-function mutation.

According to the invention, a method is provided for identifying a compound which modulates RNAi by contacting a cell containing a construct of the invention with a test compound; and determining the effect of the test compound on an indicator of RNAi activity in the cell, thereby identifying a compound which modulates RNAi. The invention also features the compound that is identified by such a method. The present invention also provides a method for modulating RNAi by contacting a cell expressing a construct of the invention with a compound in a sufficient concentration to modulate the activity of RNAi.

The invention features a method for deriving information about the function of a gene in a cell or organism by introducing into the cell or organism a construct of the invention; maintaining the cell or organism under conditions such that RNAi can occur; determining a characteristic or property of said cell or organism; and comparing said characteristic or property to a suitable control, the comparison yielding information about the function of the gene.

According to the invention, a method is provided for validating a candidate protein as a suitable target for drug discovery by introducing into a cell or organism a construct of the invention; maintaining the cell or organism under conditions such that RNAi can occur; determining a characteristic or property of the cell or organism; and comparing the characteristic or property to a suitable control, the comparison yielding information about whether the candidate protein is a suitable target for drug discovery.

The invention also features a kit containing reagents for activating RNAi in a cell or organism, a construct of the invention and instructions for use.

Other features and advantages of the invention will be apparent from the following detailed description, and from the claims.

### BRIEF DESCRIPTION OF THE DRAWINGS

FIG. 1 is a depiction of the design of hairpin constructs against mutant SOD1^{G93A}.

FIG. 1A is the nucleic acid sequence surrounding the mutation site of SOD1^{G93A} (G93A shRNA sequence, SEQ ID NO: 1; wild type SOD1 target sequence, SEQ ID NO: 2; mutant SOD1^{G93A} target sequence, SEQ ID NO: 3).

FIG. 1B is a depiction of the variations of the U6 promoter. The constructs: U6G93Ahp: authentic U6 promoter with G93A hairpin; EN-U6G93Ahp: forward CMV enhancer placed at the 5′ of the U6 promoter; REN-U6G93Ahp: reverse CMV enhancer placed at the 5′ of the U6 promoter; U6G93Ahp-EN: forward CMV enhancer placed at the 3′ of U6G93Ahp; U6G93Ahp-REN: reverse CMV enhancer placed at the 3′ of U6G93Ahp; and EN-δU6G93Ahp: forward CMV enhancer placed at the 5′ of the crippled U6 promoter with DSE deletion.

FIG. 2 is a Northern blot detecting expression of the G93Ahp transcripts.

FIGS. 3A-3B are graphs demonstrating that the CMV enhancer increases the inhibition of the target gene expression. FIG. 3A is a fluorometer measurement of GFP fluorescence in lysates from the 293 cells transfected with SOD1G93AGFP and various U6G93Ahp constructs. FIG. 3B is the average peak GFP fluorescence intensity from 6 independent experiments shown in FIG. 3A. The numbers that mark the X axis correspond to the numbers in the legend of A. Error bars indicate Standard Error of the Mean (S.E.M.).

FIG. 3C is an immunoblot of SOD1. The lane numbers correspond to the numbers in the legend of A.

### DETAILED DESCRIPTION OF THE INVENTION

The present invention provides an shRNA-expressing construct controlled by a Pol III U6 promoter (22) designed to silence a mutant allele that causes a disease or disorder in a subject. As proof of concept, the inventors of the present invention provide constructs that are designed to silence the Cu, Zn superoxide dismutase (SOD1^{G93A}) allele that causes amyotrophic lateral sclerosis (ALS), a fatal degenerative motor neuron disease (23). While testing the efficacy of this shRNA, it was found to selectively inhibit the expression of a mutant SOD1^{G93A} but did not affect SOD1^{WT} (24). It was also found that the dose of the shRNA could be increased by enhancing the Pol III promoter activity. Some snRNAs are synthesized by a Pol II; while others by a Pol III, and they share enhancer elements (25-30). Hence, a Pol II enhancer might be able to enhance Pol III driven transcription. Placing the enhancer from the CMV promoter near the U6 promoter resulted in enhanced U6 promoter activity, increased the shRNA synthesis and strengthened the silencing of the target gene. This enhanced promoter may be broadly useful in similar situations in targeting other disease-associated mutants, e.g., neurodegenerative diseases.

Technical advances in RNAi research raise the possibility that specific constructs can be designed to express shRNA in vivo to silence target genes. For example, constructs may be inserted into a virus for transducing cells in vivo. Such a strategy may become a therapeutic intervention for diseases caused by dominant, gain-of-function gene mutations. In addition, these constructs may be used to inhibit expression of genes to investigate gene function by transfection in cultured cells or by transgenic approach in vivo. The feasibility of these strategies has been demonstrated. Both viral vector- and transgene-directed synthesis of shRNA have been shown to mediate inhibition of endogenous genes in cultured cells and in vivo (Brummelkamp et al., 2002a; Hasuwa et al., 2002; Xia et al., 2002; Rubinson et al., 2003; Tiscomia et al., 2003). The possibility of RNAi therapy has been tested in several cellular and animal models of diseases. Efficacy of RNAi has been demonstrated against viral infection (Gitlin et al., 2002; Jacque et al., 2002), cancer cell proliferation (Brummelkamp et al., 2002a; Wilda et al., 2002; Cioca et al., 2003), polyglutamine diseases (Caplen et al., 2002; Xia et al., 2002), and liver fibrosis (Song, 2003). The list of diseases will undoubtedly grow in the future.

To treat dominant genetic disorders of the gain-of-function type, the mutant protein expression may be selectively silenced, thereby allowing the wild-type allele to continue functioning. Given that the vast majority of gene mutations that cause dominant diseases are single nucleotide changes, the question arises whether RNAi mediated by siRNA can discriminate mutant from the wild-type mRNA with single nucleotide specificity. Current literature presents conflicting answers to this question. siRNAs that differ from the sequence of their target RNA at one or more nucleotides retain efficacy in some cases (Boutla et al., 2001; Holen et al., 2002) and lose activity in others (Boutla et al., 2001; Elbashir et al., 2001b; Brummelkamp et al., 2002a; Brummelkamp et al., 2002b; Yu et al., 2002; Zeng and Cullen, 2003). The compositions of the present invention provide siRNAs that selectively silence the expression of mutant SOD1. The data presented herein suggest that some siRNAs can achieve single nucleotide specificity and these siRNA sequences can be found by screens using in vitro RNAi reactions and transfected cells.

The potential of using RNAi for therapy is not limited to directly silencing pathogenic genes or disease-causing mutant genes. As disease mechanisms become increasingly clear, its application can be expanded to silence genes involved in known pathogenic pathways. For example, in Alzheimer's disease where an increased Aβ peptide production and accumulation is believed to cause neuronal degeneration, an obvious target for treatment is the beta-site APP-cleaving enzyme BACE, which is required for the production of Aβ (Cai et al., 2001; Luo et al., 2001; Roberds et al., 2001). Another possible target is the γ-secretase, an enzyme complex that is made of multiple protein subunits and is also required for the production of Aβ (Haass and Steiner, 2002). One study has shown that siRNAs against different components of this enzyme complex compromise the γ-secretase function and decrease Aβ production (Lee et al., 2002b), although in vivo inhibition of γ-secretase may be complicated because γ-secretase performs other essential functions besides processing βAPP (Sisodia and St George-Hyslop, 2002). These possible applications illustrate that RNAi may be used not only to treat familial diseases with identified dominant gene mutations, but also to treat sporadic diseases.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

I. Definitions

So that the invention may be more readily understood, certain terms are first defined.

The term “DNA segment” refers to a linear fragment of single- or double-stranded deoxyribonucleic acid (DNA), which can be derived from any source.

As used herein, the term “encodes” means the generation of a RNA molecule from a DNA molecule (i.e., a complementary RNA molecule generated from the DNA molecule by the process of transcription) or the generation of a polypeptide or protein molecule from a DNA molecule via a RNA intermediate (i.e., by the processes of transcription and translation).

The term “construct”, as used herein refers to an engineered DNA molecule including one or more nucleotide sequences from different sources. A preferred construct includes at least a shRNA-encoding region operably linked to a promoter sequence.

The term “enhancer” refers to a DNA sequence which, when bound by a specific protein factor, enhances the levels of expression of a gene, but is not sufficient alone to cause expression. An “enhancer” is capable of enhancing expression of a gene regardless of the distance from the gene or orientation relative to the gene.

The term “kit” is any manufacture (e.g. a package or container) comprising at least one reagent, e.g. a construct, for activating RNAi in a cell or organism, the manufacture being promoted, distributed, or sold as a unit for performing the methods of the present invention.

The term “gene” includes cDNAs, RNA, or other polynucleotides that encode gene products. “Foreign gene” denotes a gene that has been obtained from an organism or cell type other than the organism or cell type in which it is expressed; it also refers to a gene from the same organism that has been translocated from its normal situs in the genome.

The term “target gene”, as used herein, refers to a gene intended for downregulation via RNA interference (“RNAi”). The term “target protein” refers to a protein intended for downregulation via RNAi. The term “target RNA” refers to an RNA molecule intended for degradation by RNAi. An exemplary “target RNA” is a coding RNA molecule (i.e., a mRNA molecule).

The term “promoter” refers to a DNA sequence to which RNA polymerase can bind and initiate transcription. An “inducible promoter” is a DNA sequence which, when operably linked with a DNA sequence encoding a specific gene product, causes the gene product to be substantially produced in a cell only when an inducer which corresponds to the promoter is present in the cell. The term “Pol III promoter” refers to an RNA polymerase III promoter. Exemplary Pol III promoters include, but are not limited to, the U6 promoter, the H1 promoter, and the tRNA promoters. The term “Pol II promoter” refers to an RNA polymerase II promoter. Exemplary Pol II promoters include, but are not limited to, the CMV promoter and the Ubiquitin C promoter.

The term “expression” of a gene or nucleic acid encompasses not only cellular gene expression, but also the transcription and translation of nucleic acid(s) in cloning systems and in any other context.

The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNA agent”, as used herein, refers to an RNA (or analog thereof), comprising a sequence having sufficient complimentarity to a target RNA (i.e., the RNA being degraded) to direct RNAi. A sequence having a “sufficiently complementary to a target RNA sequence to direct RNAi” means that the RNA agent has a sequence sufficient to trigger the destruction of the target RNA by the RNAi machinery (e.g., the RISC complex) or process.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term “mRNA” or “messenger RNA” refers to a single-stranded RNA that specifies the amino acid sequence of one or more polypeptide chains. This information is translated during protein synthesis when ribosomes bind to the mRNA.

The term “neurological” disease or disorder means a disease or disorder effecting the nervous system. Exemplary neurodegenerative diseases include Lou Gehrig's disease, amyotrophic lateral sclerosis (ALS), Alzheimer's disease, Parkinson's disease, Adrenoleukodystrophy (ALD), and dementia.

The term “gene product” refers primarily to proteins and polypeptides encoded by other nucleic acids (e.g., non-coding and regulatory RNAs such as tRNA, sRNPs). The term “regulation of expression” refers to events or molecules that increase or decrease the synthesis, degradation, availability or activity of a given gene product.

The term “transcript” refers to a RNA molecule transcribed from a DNA or RNA template by a RNA polymerase template. The term “transcript” includes RNAs that encode polypeptides (i.e., mRNAs) as well as noncoding RNAs (“ncRNAs”).

As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example, 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., the RISC complex).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term “subject”, as used herein, includes living organisms at risk for or having a cell neurological, e.g. neurodegenerative disease or disorder. Examples of subjects include humans, monkeys, cows, sheep, goats, dogs, cats, mice, rats, and transgenic species thereof. Administration of the compositions of the present invention to a subject to be treated can be carried out using known procedures, at dosages and for periods of time effective to modulate RNAi in the subject as further described herein.

The term “treatment”, as used herein, is defined as the application or administration of a therapeutic agent to a subject, or application or administration of a therapeutic agent to an isolated tissue or cell line from a subject, who has a disease or disorder, a symptom of a disease or disorder, or a predisposition toward a disease or disorder, with the purpose to cure, heal, alleviate, relieve, alter, remedy, ameliorate, improve or affect the disease or disorder, the symptoms of the disease or disorder, or the predisposition toward a disease or disorder. A therapeutic agent includes, but is not limited to, small molecules, peptides, antibodies, ribozymes, antisense oligonucleotides, chemotherapeutic agents and radiation.

The term “effective amount”, as used here in, is defined as that amount necessary or sufficient to treat or prevent a disorder, e.g. a neurological or a neurodegenerative disease or disorder. The effective amount can vary depending on such factors as the size and weight of the subject, the type of illness, or the particular agent being administered. One of ordinary skill in the art would be able to study the aforementioned factors and make the determination regarding the effective amount of the agent without undue experimentation.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “mutation” refers to a substitution, addition, or deletion of a nucleotide within a gene sequence resulting in aberrant production (e.g., misregulated production) of the protein encoded by the gene sequence. A “gain-of-function” mutation is a mutation that results in production of a protein having aberrant function as compared to the wild-type or normal protein encoded by a gene sequence.

The term “pharmaceutical composition” as used herein, refers to an agent formulated with one or more compatible solid or liquid filler diluents or encapsulating substances which are suitable for administration to a human or lower animal.

A gene “involved” in a disorder includes a gene, the normal or aberrant expression or function of which effects or causes a disease or disorder or at least one symptom of said disease or disorder

The phrase “examining the function of a gene in a cell or organism” refers to examining or studying the expression, activity, function or phenotype arising therefrom.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

The term “upstream” refers to nucleotide sequences that precede, e.g., are on the 5′ side of, a reference sequence.

The term “downstream” refers to nucleotide sequences that follow, e.g., are on the 3′ side of, a reference sequence.

The terms used herein are not intended to be limiting of the invention.

II. shRNA-Encoding Nucleic Acids

Preferred constructs of the instant invention include nucleic acid sequences or molecules that encode (i.e., generate) shRNA molecules. The requisite elements of a shRNA-encoding nucleic acid sequence or molecule include a first portion and a second portion, having sequences such that the RNA sequences encoded by said portions have sufficient complementarity to anneal or hybridize to form a duplex or double-stranded stem portion. The two portions need not be fully or perfectly complementary. The first and second “stem-encoding” portions are connected by a portion having a sequence that, when encoded, has insufficient sequence complementarity to anneal or hybridize to other portions of the shRNA. This latter portion is referred to as a “loop-encoding” portion in the shRNA-encoding nucleic acid sequences or molecules. The shRNA-encoding nucleic acid sequences or molecules are transcribed to generate shRNAs. shRNAs can also include one or more bulges, i.e., extra nucleotides that create a small nucleotide “loop” in a portion of the stem, for example a one-, two- or three-nucleotide loop. The encoded stem portions can be the same length, or one portion can include an overhang of, for example, 1-5 nucleotides. The overhanging nucleotides can include, for example, uracils (Us), e.g., all Us. Such Us are notably encoded by thymidines (Ts) in the shRNA-encoding DNA which signal the termination of transcription.

One strand of the stem portion of the encoded shRNA is further sufficiently complementary (e.g., antisense) to a target RNA (e.g., mRNA) sequence to mediate degradation or cleavage of said target RNA via RNA interference (RNAi). The antisense portion can be on the 5′ or 3′ end of the stem. The stem-encoding portions of a shRNA-encoding nucleic acid (or stem portion of a shRNA) are preferably about 15 to about 50 nucleotides in length. When used in mammalian cells, the length of the stem portions should be less than about 30 nucleotides to avoid provoking non-specific responses like the interferon pathway. In non-mammalian cells, the stem can be longer than 30 nucleotides. In fact, a stem portion can include much larger sections complementary to the target mRNA (up to, and including the entire mRNA). The loop portion in the shRNA (or loop-encoding portion in the encoding DNA) can be about 2 to about 20 nucleotides in length, i.e., about 2, 3, 4, 5, 6, 7, 8, 9, or more, e.g., 15 or 20, or more nucleotides in length. A preferred loop consists of or comprises a “tetraloop” sequences. Exemplary tetraloop sequences include, but are not limited to, the sequences GNRA, where N is any nucleotide and R is a purine nucleotide, GGGG, and UUUU.

The sequence of the antisense portion of a shRNA can be designed by selecting an 18, 19, 20, 21 nucleotide, or longer, sequence from within the target RNA (e.g., mRNA), for example, from a region 100 to 200 or 300 nucleotides upstream or downstream of the start of translation. In general, the sequence can be selected from any portion of the target RNA (e.g., mRNA) including the 5′ UTR (untranslated region), coding sequence, or 3′ UTR. This sequence can optionally follow immediately after a region of the target gene containing two adjacent AA nucleotides. The last two nucleotides of the nucleotide sequence can be selected to be UU. shRNAs so generated are processed under appropriate conditions (e.g., in an appropriate in vitro reaction or in a cell) by RNAi machinery (i.e., Dicer and/or RISC complexes) to generate siRNAs. shRNAs can be synthesized exogenously or can be transcriped in vivo from an RNA polymerase (e.g., a Pol II or Pol III polymerase), thus permitting the construction of continuous cell lines or transgenic animals in which the desired gene silencing is stable and heritable.

In certain aspects of the invention, it may be important to detect the generation or expression of shRNAs, target mRNAs and/or the gene products encoded by said target RNAs. The detection methods used herein include, for example, cloning and sequencing, ligation of oligonucleotides, use of the polymerase chain reaction and variations thereof (e.g., a PCR that uses 7-deaza GTP), use of single nucleotide primer-guided extension assays, hybridization techniques using target-specific oligonucleotides that can be shown to preferentially bind to complementary sequences under given stringency conditions, and sandwich hybridization methods.

Sequencing may be carried out with commercially available automated sequencers utilizing labeled primers or terminators, or using sequencing gel-based methods. Sequence analysis is also carried out by methods based on ligation of oligonucleotide sequences which anneal immediately adjacent to each other on a target DNA or RNA molecule (Wu and Wallace, Genomics 4: 560-569 (1989); Landren et al., Proc. Natl. Acad. Sci. 87: 8923-8927 (1990); Barany, F., Proc. Natl. Acad. Sci. 88: 189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating late onset diabetes mutation loci. The elevated reaction temperatures permits the ligation reaction to be conducted with high stringency (Barany, F., PCR Methods and Applications 1: 5-16 (1991)).

The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

Detection oligonucleotide probes range in size between 10-1,000 bases. In order to obtain the required target discrimination using the detection oligonucleotide probes, the hybridization reactions are generally run between 20°-60° C., and most preferably between 30°-50° C. As known to those skilled in the art, optimal discrimination between perfect and mismatched duplexes is obtained by manipulating the temperature and/or salt concentrations or inclusion of formamide in the stringency washes.

Detection of proteins may be carried out using specific antibodies, e.g., monoclonal or polyclonal antibodies, or fragments thereof.

Preferred detection reagents are labeled, e.g., fluorescents, coloro-metrically or radio-iso-typically labeled to facilitate visulalization and/or quantitation.

III. Constructs/Transgene

A construct is a recombinant nucleic acid, generally recombinant DNA, generated for the purpose of the expression of a specific nucleotide sequence(s), or is to be used in the construction of other recombinant nucleotide sequences. In particular, a construct of the invention comprises a nucleotide sequence encoding a small hairpin RNA (shRNA) under the transcriptional control of a modified promoter. Promoters useful in constructs of the invention include Pol III promoters, e.g., a U6 promoter, H1 promoter or tRNA promoter, and Pol II promoters, e.g., a CMV promoter, which may be used to increase the potency of shRNA by increasing the expression level. Promoters of the invention are preferably upstream of the shRNA encoding sequence and are at a distance sufficient so that the shRNA is expressed. Promoters can be, for example, within 2 kb, 1 kb, 750 bp, 500 bp, 400 bp, 300 bp, 200 bp, 100 bp or 50 bp of the shRNA encoding sequence.

Modifications to the promoter include the presence of an enhancer. An enhancer can be upstream of the promoter, e.g., upstream of the promoter and the shRNA encoding sequence. An enhancer can alternatively be downstream of the shRNA encoding sequence, e.g., downstream of the promoter and the shRNA encoding sequence. An enhancer in constructs of the invention is at a distance from the promoter such that expression of the shRNA is enhanced or increased. In constructs of the invention, enhancers can be, for example, within 5 kb, 2.5 kb, 2 kb, 1.5 kb, 1 kb, 0.5 kb, 0.1 kb or less from the promoter. Enhancers can be in either forward or backward orientation. In particular, constructs of the invention comprising modified promoters include constructs with a Pol II enhancer, such as the cytomegalovirus (CMV) enhancer, immediate-early promoter near the Pol III, e.g., U6 promoter, either upstream or downstream from the shRNA sequence and in either forward or backward orientation. Such constructs are useful for increasing the expression of the shRNA, thereby enhancing inhibition of a single nucleotide mismatched mutant allele. An enhancer useful in the invention is capable of stimulating or enhancing expression of the shRNA by about 2-fold, 5-fold, 10-fold, 25-fold, 50-fold, 75-fold or 100-fold or more. A transgene is a construct that has been or is designed to be incorporated into a cell, particularly a mammalian cell, that in turn becomes or is incorporated into a living animal such that the construct containing the nucleotide sequence is expressed (i.e., the mammalian cell is transformed with the transgene). The transgene includes a sequence (e.g., a shRNA-encoding sequence) that is endogenous to the transgenic animal. A transgene may be present as an extrachromosomal element in some or all of the cells of a transgenic animal or, preferably, stably integrated into some or all of the cells, more preferably into the germline DNA of the animal (i.e., such that the transgene is transmitted to all or some of the animal's progeny), thereby directing expression of the product of the transgene in one or more cell types or tissues of the transgenic animal. Unless otherwise indicated, it will be assumed that a transgenic animal comprises stable changes to the chromosomes of germline cells. In a preferred embodiment, the transgene is present in the genome at a site such that it does not interfere with gene expression.

Such transgenic animals are created by introducing a transgenic construct of the invention into its genome using methods and vectors as described herein.

A transgenic construct of the invention includes the encoding sequence operably linked to an appropriate promoter sequence. The transgene optionally includes enhancer sequences and other non-coding sequences (for example, intron and/or 5′ or 3′ untranslated sequences).

IV. Vectors and Host Cells

Another aspect of the invention pertains to vectors, preferably expression vectors, containing a construct of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

In general, expression vectors of utility in recombinant DNA techniques are often in the form of plasmids. Accordingly, in one embodiment, an expression vector of the invention is a plasmid. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Thus, in one embodiment, an expression vector of the invention is a viral-based vector. For example, replication defective retroviruses, adenoviruses and adeno-associated viruses can be used. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include ψCrip, ψCre, ψ2 and ψAm. The genome of adenovirus can be manipulated such that it encodes and expresses a regulatable shRNA construct, as described herein, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Alternatively, an adeno-associated virus vector such as that described in Tratschin et al. (1985) Mol. Cell. Biol. 5:3251-3260 can be used to express a transactivator fusion protein. In a particular embodiment of the invention, an expression vector is not a viral vector.

The vectors of the invention comprise a shRNA-encoding nucleic acid operatively linked to one or more regulatory sequences (e.g., promoter sequences, e.g., Pol II or Pol III promoter sequences). The phrase “operably linked” is intended to mean that the nucleotide sequence of interest (e.g., the shRNA-encoding sequence) is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Other elements included in the design of a particular expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The vectors described herein can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference. See, also, Ausubel et al., Current Protocols in Molecular Biology, John Wiley and Sons, Baltimore, Md. (1989); Hitt et al, “Construction and propagation of human adenovirus vectors,” in Cell Biology: A Laboratory Handbook, Ed. J. E. Celis., Academic Press. 2.sup.nd Edition, Volume 1, pp: 500-512, 1998; Hitt et al, “Techniques for human adenovirus vector construction and characterization,” in Methods in Molecular Genetics, Ed. K. W. Adolph, Academic Press, Orlando, Fla., Volume 7B, pp:12-30, 1995; Hitt, et al., “Construction and propagation of human adenovirus vectors,” in Cell Biology: A Laboratory Handbook,” Ed. J. E. Celis. Academic Press. pp:479-490, 1994, also hereby incorporated by reference. The methods include, for example, stable or transient transfection, lipofection, electroporation and infection with recombinant viral vectors. The term “transfecting” or “transfection” is intended to encompass all conventional techniques for introducing nucleic acid into host cells, including calcium phosphate co-precipitation, DEAE-dextran-mediated transfection, lipofection, electroporation and microinjection. Suitable methods for transfecting host cells can be found in Sambrook et al. (Molecular Cloning: A Laboratory Manual, 2nd Edition, Cold Spring Harbor Laboratory press (1989)), and other laboratory textbooks.

The number of host cells transformed with a nucleic acid of the invention will depend, at least in part, upon the type of recombinant expression vector used and the type of transfection technique used. Nucleic acid can be introduced into a host cell transiently, or more typically, for long term regulation of gene expression, the nucleic acid is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acid of interest. Preferred selectable markers include those which confer resistance to certain drugs, such as G418 and hygromycin. Selectable markers can be introduced on a separate plasmid from the nucleic acid of interest or, are introduced on the same plasmid. Host cells transfected with a nucleic acid of the invention (e.g., a recombinant expression vector) and a gene for a selectable marker can be identified by selecting for cells using the selectable marker. For example, if the selectable marker encodes a gene conferring neomycin resistance, host cells which have taken up nucleic acid can be selected with G418. Cells that have incorporated the selectable marker gene will survive, while the other cells die.

Nucleic acid encoding a regulatable shRNA of the invention can be introduced into eukaryotic cells growing in culture in vitro by conventional transfection techniques (e.g., calcium phosphate precipitation, DEAE-dextran transfection, electroporation etc.). Nucleic acid can also be transferred into cells in vivo, for example by application of a delivery mechanism suitable for introduction of nucleic acid into cells in vivo, such as retroviral vectors (see e.g., Ferry, N et al. (1991) Proc. Natl. Acad. Sci. USA 88:8377-8381; and Kay, M. A. et al. (1992) Human Gene Therapy 3:641-647), adenoviral vectors (see e.g., Rosenfeld, M. A. (1992) Cell 68:143-155; and Herz, J. and Gerard, R. D. (1993) Proc. Natl. Acad. Sci. USA 90:2812-2816), receptor-mediated DNA uptake (see e.g., Wu, G. and Wu, C. H. (1988) J. Biol. Chem. 263:14621; Wilson et al. (1992) J. Biol. Chem. 267:963-967; and U.S. Pat. No. 5,166,320), direct injection of DNA (see e.g., Acsadi et al. (1991) Nature 332: 815-818; and Wolff et al. (1990) Science 247:1465-1468) or particle bombardment (see e.g., Cheng, L. et al. (1993) Proc. Natl. Acad. Sci. USA 90:4455-4459; and Zelenin, A. V. et al. (1993) FEBS Letters 315:29-32). Thus, for gene therapy purposes, cells can be modified in vitro and administered to a subject or, alternatively, cells can be directly modified in vivo.

Another aspect of the invention pertains to host cells into which a host construct of the invention has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells (such as Chinese hamster ovary cells (CHO) or COS cells). Other suitable host cells are known to those skilled in the art.

The host cells of the invention can also be used to produce nonhuman transgenic animals. The nonhuman transgenic animals can be used in screening assays designed to identify agents or compounds, e.g., drugs, pharmaceuticals, etc., which are capable of ameliorating detrimental symptoms of selected disorders, such as disease and disorders associated with mutant or aberrant gene expression, gain-of-function mutants and neurological diseases and disorders.

The present invention is also not limited to the use of the cell types and cell lines used herein. Cells from different tissues or different species (human, mouse, etc.) are also useful in the present invention.

V. Construction of Transgenic Animals

In one aspect, the present invention provides a non-human animal whose genome contains a shRNA-encoding construct or transgene of the invention. The present invention further provides methods for making a transgenic non-human animal whose genome contains a shRNA-encoding construct or transgene of the invention.

The transgenic animal used in the methods of the invention can be, e.g., a mammal, a bird, a reptile or an amphibian. Suitable mammals for uses described herein include: rodents; ruminants; ungulates; domesticated mammals; and dairy animals. Preferred animals include: rodents, goats, sheep, camels, cows, pigs, horses, oxen, llamas, chickens, geese, and turkeys. In a preferred embodiment, the non-human animal is a mouse.

Various methods of making transgenic animals are known in the art (see, e.g., Watson, J. D., et al., “The Introduction of Foreign Genes Into Mice,” in Recombinant DNA, 2d Ed., W. H. Freeman & Co., New York (1992), pp. 255-272; Gordon, J. W., Intl. Rev. Citole. 115:171-229 (1989); Janis, R., Science 240: 1468-1474 (1989); Ross ant, J., Neuron 2: 323-334 (1990)). An exemplary protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. An exemplary protocol for the production of a transgenic rat can be found in Bader and Ganten, Clinical and Experimental Pharmacology and Physiology, Supp. 3:S81-S87, 1996. An exemplary protocol for the production of a transgenic cow can be found in Transgenic Animal Technology, A Handbook, 1994, ed., Carl A. Pinkert, Academic Press, Inc. An exemplary protocol for the production of a transgenic sheep can be found in Transgenic Animal Technology, A Handbook, 1994, ed., Carl A. Pinkert, Academic Press, Inc. Several exemplary methods are set forth in more detail below.

A. Injection into the Pronucleus

Transgenic animals can be produced by introducing a nucleic acid construct according to the present invention into egg cells. The resulting egg cells are implanted into the uterus of a female for normal fetal development, and animals which develop and which carry the transgene are then backcrossed to create heterozygotes for the transgene. Embryonal target cells at various developmental stages are used to introduce the transgenes of the invention. Different methods are used depending on the stage of development of the embryonal target cell(s). Exemplary methods for introducing transgenes include, but are not limited to, microinjection of fertilized ovum or zygotes (Brinster, et al., Proc. Natl. Acad. Sci. USA (1985) 82: 4438-4442), and viral integration (Jaenisch R., Proc. Natl. Acad. Sci. USA (1976) 73: 1260-1264; Jahner, et al., Proc. Natl. Acad. Sci. USA (1985) 82: 6927-6931; Van der Putten, et al., (1985) Proc. Natl. Acad. Sci. (USA) 82: 6148-6152). Procedures for embryo manipulation and microinjection are described in, for example, Manipulating the Mouse Embryo (Cold Spring Harbor Laboratory Press, Cold Spring Harbor, N.Y., 1986, the contents of which are incorporated herein by reference). Similar methods are used for production of other transgenic animals.

In an exemplary embodiment, production of transgenic mice employs the following steps. Male and female mice, from a defined inbred genetic background, are mated. The mated female mice are previously treated with pregnant mare serum, PMS, to induce follicular growth and human chorionic gonadotropin, hCG, to induce ovulation. Following mating, the female is sacrificed and the fertilized eggs are removed from her uterine tubes. At this time, the pronuclei have not yet fused and it is possible to visualize them using light microscopy. In an alternative protocol, embryos can be harvested at varying developmental stages, e.g. blastocysts can be harvested. Embryos are recovered in a Dulbecco's modified phosphate buffered saline (DPBS) and maintained in Dulbecco's modified essential medium (DMEM) supplemented with 10% fetal bovine serum.

Foreign DNA or the recombinant construct (e.g. shRNA-encoding construct or transgene) is then microinjected (100-1000 molecules per egg) into a pronucleus. Microinjection of an expression construct can be performed using standard micro manipulators attached to a microscope. For instance, embryos are typically held in 100 microliter drops of DPBS under oil while being microinjected. DNA solution is microinjected into the male pronucleus. Successful injection is monitored by swelling of the pronucleus. Shortly thereafter, fusion of the pronuclei (a female pronucleus and a male pronucleus) occurs and, in some cases, foreign DNA inserts into (usually) one chromosome of the fertilized egg or zygote. Recombinant ES cells, which are prepared as set forth below, can be injected into blastocysts using similar techniques.

B. Embryonic Stem Cells

In another method of making transgenic mice, recombinant DNA molecules (e.g., constructs or transgenes) of the invention can be introduced into mouse embryonic stem (ES) cells. Resulting recombinant ES cells are then microinjected into mouse blastocysts using techniques similar to those set forth in the previous subsection.

ES cells are obtained from pre-implantation embryos and cultured in vitro (Evans, M J., et al., Nature 292: 154156 (1981); Bradley, M. O. et al., Nature 309: 255-258 (1984); Gossler, et al., Proc. Natl. Acad. Sci. (USA) 83:9065-9069 (1986); Robertson et al., Nature 322: 445448 (1986)). Any ES cell line that is capable of integrating into and becoming part of the germ line of a developing embryo, so as to create germ line transmission of the targeting construct, is suitable for use herein. For example, a mouse strain that can be used for production of ES cells is the 129J strain. A preferred ES cell line is murine cell line D3 (American Type Culture Collection catalog no. CRL 1934). The ES cells can be cultured and prepared for DNA insertion using methods known in the art and described in Robertson, Teratocarcinomas and Embryonic Stem Cells: A Practical Approach, E. J. Robertson, ed. IRL Press, Washington, D.C., 1987, in Bradley et al., Current Topics in Devel. Biol., 20:357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The expression construct can be introduced into the ES cells by methods known in the art, e.g., those described in Sambrook et al., Molecular Cloning: A Laboratory Manual, 2^{nd} Ed., ed., Cold Spring Harbor laboratory Press: 1989, the contents of which are incorporated herein by reference. Suitable methods include, but are not limited to, electroporation, microinjection, and calcium phosphate treatment methods. The foreign DNA (e.g. construct or transgene) to be introduced into the ES cell is preferably linear.

After introduction of the expression construct, the ES cells are screened for the presence of the construct. The cells can be screened using a variety of methods. ES cell genomic DNA can be examined directly. For example, the DNA can be extracted from the ES cells using standard methods and the DNA can then be probed on a Southern blot with a probe or probes designed to hybridize specifically to the transgene. The genomic DNA can also be amplified by PCR with probes specifically designed to amplify DNA fragments of a particular size and sequence of the construct or transgene such that, only those cells containing the construct or transgene will generate DNA fragments of the proper size. Where a marker gene is employed in the construct, the cells of the animal can be tested for the presence of the marker gene. For example, where the marker gene is an antibiotic resistance gene, the cells can be cultured in the presence of an otherwise lethal concentration of antibiotic (e.g. G418 to select for neo). Those cells that survive have presumably integrated the transgene construct. If the marker gene is a gene that encodes an enzyme whose activity can be detected (e.g., β-galactosidase), the enzyme substrate can be added to the cells under suitable conditions, and the enzymatic activity can be analyzed.

C. Implantation

The zygote harboring a recombinant nucleic acid molecule of the invention (e.g. construct or transgene) is implanted into a pseudo-pregnant female mouse that was obtained by previous mating with a vasectomized male. In a general protocol, recipient females are anesthetized, paralumbar incisions are made to expose the oviducts, and the embryos are transformed into the ampullary region of the oviducts. The body wall is sutured and the skin closed with wound clips. The embryo develops for the full gestation period, and the surrogate mother delivers the potentially transgenic mice. Finally, the newborn mice are tested for the presence of the foreign or recombinant DNA. Of the eggs injected, on average 10% develop properly and produce mice. Of the mice born, on average one in four (25%) are transgenic for an overall efficiency of 2.5%. Once these mice are bred they transmit the foreign gene in a normal (Mendelian) fashion linked to a mouse chromosome.

D. Screening for the Presence of the Transgenic Construct

Transgenic animals can be identified after birth by standard protocols. DNA from tail tissue can be screened for the presence of the transgene construct, e.g., using southern blots and/or PCR. Offspring that appear to be mosaics are then crossed to each other if they are believed to carry the transgene in order to generate homozygous animals. If it is unclear whether the offspring will have germ line transmission, they can be crossed with a parental or other strain and the offspring screened for heterozygosity. The heterozygotes are identified by southern blots and/or PCR amplification of the DNA. The heterozygotes can then be crossed with each other to generate homozygous transgenic offspring. Homozygotes may be identified by southern blotting of equivalent amounts of genomic DNA from mice that are the product of this cross, as well as mice that are known heterozygotes and wild type mice. Probes to screen the southern blots can be designed based on the sequence of the construct or transgene, or a marker gene, or both.

Other means of identifying and characterizing the transgenic offspring are known in the art. For example, western blots can be used to assess the level of expression of a gene targeted for interference by probing the with an antibody against the protein encoded by the target gene. Alternatively, an antibody against a marker gene product can be used, when a marker gene is expressed.

E. Mice Containing Multiple Transgenes

Transgenic mice expressing shRNAs as described herein can be crossed with mice that harbor additional transgene(s). Mice that are heterozygous or homozygous for shRNA expression can be generated and maintained using standard crossbreeding procedures.

The invention further pertains to cells derived from transgenic animals. Because certain modifications may occur in succeeding generations due to either mutation or environmental influences, such progeny may not, in fact, be identical to the parent cell, but are still included within the scope of the term as used herein.

VI. Uses and Methods of the Invention

The methods of the present invention will find great commercial application, for example in biotechnology, drug development and medicine. For example, in biotechnology, the ability to rapidly develop large numbers of transgenic animals with desired modulation of specific genes will allow for the analysis of gene function and the evaluation of compounds that potentially modulate gene expression, protein function, and are useful in treating a disease or disorder. In particular, by observing the effect of down-regulating specific genes in transgenic animals, the biological function of those genes may be determined. In medicine the methods of the invention may be used to treat patients suffering from particular diseases or disorders, for example, neurological diseases or disorders, or to confer immunity or resistance to particular pathogens. For example, specific cells may be infected in vivo or ex vivo with recombinant retrovirus encoding an siRNA that down-regulates the activity of a gene whose activity is associated with a particular disease or disorder.

A. Screening Assays

Cells and/or animals of the present invention may also be suitable for use in methods to identify and/or characterize potential pharmacological agents, e.g. identifying new pharmacological agents from a collection of test substances and/or characterizing mechanisms of action and/or side effects of known pharmacological agents.

Thus, the present invention also relates to a system for identifying and/or characterizing pharmacological agents comprising: (a) a cell (e.g., a eukaryotic cell) or organism (e.g., a eukaryotic non-human organism) containing a construct or transgene of the invention and (b) a test substance or a collection of test substances wherein pharmacological properties of said test substance or said collection are to be identified and/or characterized. Optionally, the system as described above can further comprise suitable controls.

Test compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

Compounds or agents identified according to such screening assays can be used therapeutically or prophylactically either alone or in combination, for example, with an shRNA of the invention, as described herein.

B. Knockout and/or Knockdown Cells or Organisms

A shRNAs (either known or identified by the methodologies of the present invention) can be used in a functional analysis of the corresponding target RNA (either known or identified by the methodologies of the present invention). Such a functional analysis is typically carried out in eukaryotic cells, or eukaryotic non-human organisms, preferably mammalian cells or organisms and most preferably human cells, e.g. cell lines such as HeLa or 293 or rodents, e.g. rats and mice. By administering a suitable shRNA molecule, a specific knockout or knockdown phenotype can be obtained in a target cell, e.g. in cell culture or in a target organism.

Thus, further subject matter of the invention includes cells (e.g., eukaryotic cells) or organisms (e.g., eukaryotic non-human organisms) exhibiting a target gene-specific knockout or knockdown phenotype resulting from a fully or at least partially deficient expression of at least one endogeneous target gene wherein said cell or organism is transfected with or administered, respectively, at least one shRNA, vector comprising DNA encoding said shRNA (or an shRNA precursor) capable of inhibiting the expression of the target gene. It should be noted that the present invention allows a target-specific knockout or knockdown of several different endogeneous genes based on the specificity of the shRNA(s) transfected or administered.

Gene-specific knockout or knockdown phenotypes of cells or non-human organisms, particularly of human cells or non-human mammals may be used in analytic procedures, e.g. in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g. an affinity tag, particularly a multiple affinity tag.

Variants or mutated forms of the target gene differ from the endogeneous target gene in that they encode a gene product which differs from the endogeneous gene product on the amino acid level by substitutions, insertions and/or deletions of single or multiple amino acids. The variants or mutated forms may have the same biological activity as the endogeneous target gene. On the other hand, the variant or mutated target gene may also have a biological activity, which differs from the biological activity of the endogeneous target gene, e.g. a partially deleted activity, a completely deleted activity, an enhanced activity etc. The complementation may be accomplished by compressing the polypeptide encoded by the endogeneous nucleic acid, e.g. a fusion protein comprising the target protein and the affinity tag and the double stranded RNA molecule for knocking out the endogeneous gene in the target cell. This compression may be accomplished by using a suitable expression vector expressing both the polypeptide encoded by the endogenous nucleic acid, e.g. the tag-modified target protein and the double stranded RNA molecule or alternatively by using a combination of expression vectors. Proteins and protein complexes which are synthesized de novo in the target cell will contain the exogenous gene product, e.g., the modified fusion protein. In order to avoid suppression of the exogenous gene product by the siRNAi molecule, the nucleotide sequence encoding the exogenous nucleic acid may be altered at the DNA level (with or without causing mutations on the amino acid level) in the part of the sequence which so is homologous to the siRNA molecule. Alternatively, the endogeneous target gene may be complemented by corresponding nucleotide sequences from other species, e.g. from mouse.

C. Functional Genomics and/or Proteomics

Preferred applications for the cell or organism of the invention include the analysis of gene expression profiles and/or proteomes. In an especially preferred embodiment an analysis of a variant or mutant form of one or several target proteins is carried out, wherein said variant or mutant forms are reintroduced into the cell or organism by an exogenous target nucleic acid as described above. The combination of knockout of an endogeneous gene and rescue by using mutated, e.g. partially deleted exogenous target has advantages compared to the use of a knockout cell. Further, this method is particularly suitable for identifying functional domains of the targeted protein. In a further preferred embodiment a comparison, e.g. of gene expression profiles and/or proteomes and/or phenotypic characteristics of at least two cells or organisms is carried out. These organisms are selected from: (i) a control cell or control organism without target gene inhibition, (ii) a cell or organism with target gene inhibition and (iii) a cell or organism with target gene inhibition plus target gene complementation by an exogenous target nucleic acid.

Furthermore, the RNA knockout complementation method may be used for its preparative purposes, e.g. for the affinity purification of proteins or protein complexes from eukaryotic cells, particularly mammalian cells and more particularly human cells. In this embodiment of the invention, the exogenous target nucleic acid preferably codes for a target protein which is fused to art affinity tag. This method is suitable for functional proteome analysis in mammalian cells, particularly human cells. Another utility of the present invention could be a method of identifying gene function in an organism comprising the use of shRNA to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like.

The ease with which RNA can be introduced into an intact cell/organism containing the target gene allows the present invention to be used in high throughput screening (HTS). Solutions containing shRNAs that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified RNA can be fed directly to, injected into, the cell/organism containing the target gene. Alternatively, the shRNA can be produced from a vector, as described herein. Vectors can be injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a colorimetric, fluorogenic, or luminescent signal in response to a regulated promoter (e.g., transfected with a reporter gene construct) can be assayed in an HTS format.

The HTS approach may identify new drug targets. The potential drug targets may also be validated using the present invention. For example, a particular disease phenotype might be induced by a gene mutation or a chemical. RNAi may be used to down-regulate genes and some of these down-regulations might lead to the reversal of the disease phenotype. These genes are potential drug targets. Compounds may be identified to inhibit these genes to treat the disease phenotype.

D. Viral Delivery Vehicles

One challenge that must be met to realize therapeutic applications of RNAi technologies is the development of systems to deliver siRNAs efficiently into mammalian cells. Towards that end, plasmids have been designed expressing short hairpin RNAs, or stem-loop RNA structures, driven by RNA polymerase III (pol III) promoters (T. R. Brummelkamp et al. Science (2002) 296:550-553; P. J. Paddison et al., Genes Dev. (2002) 16:948-958). The hairpin RNAs are processed to generate siRNAs in cells and thereby induce gene silencing. Pol III promoters are advantageous because their transcripts are not necessarily post-transcriptionally modified, and because they are highly active when introduced in mammalian cells. Polymerase II (pol II) promoters may offer advantages to pol III promoters, including being more easily incorporated into viral expression vectors, such as retroviral and adeno-associated viral vectors, and the existence of inducible and tissue specific pol II dependent promoters.

The limitation of plasmid-based siRNA delivery systems is their dependence on cell transfection methods, which are rarely efficient and limited primarily to established cell lines. Viral based strategies would offer the significant advantage of allowing for efficient delivery to cell lines as well as primary cells.

E. Methods of Treatment

The present invention provides shRNA-expressing constructs that are useful clinically (e.g., in certain prophylactic and/or therapeutic applications). For example, shRNAs can be used, for example, as prophylactic and/or therapeutic agents in the treatment of diseases or disorders associated with unwanted or aberrant expression of the corresponding target gene.

In one embodiment, the invention provides for prophylactic methods of treating a subject at risk of (or susceptible to) a disease or disorder, for example, a disease or disorder associated with aberrant or unwanted target gene expression or activity. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

In another embodiment, the invention provides for therapeutic methods of treating a subject having a disease or disorder, for example, a disease or disorder associated with aberrant or unwanted target gene expression or activity. In an exemplary embodiment, the modulatory method of the invention involves contacting a cell capable of expressing target gene with a therapeutic agent that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

“Treatment”, or “treating” as used herein, is defined as the application or administration of a prophylactic or therapeutic agent to a patient, or application or administration of a prophylactic or therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

Knowledge of shRNAs and their targets would allow specific modulation of shRNA systems to treat any of a number of disorders (including cancer, inflammation, neuronal disorders, etc.). Manipulating shRNA regulation of translation of these genes is a novel, powerful, and specific method for treating these disorders.

VII. Pharmacogenomics and Pharmaceutical Compositions

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

With regards to the above-described agents for prophylactic and/or therapeutic treatments, the agents are routinely incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

When administering shRNAs, it may be advantageous to chemically modify the shRNA in order to increase in vivo stability. Preferred modifications stabilize the shRNA against degradation by cellular nucleases.

The pharmaceutical compositions can be included in a container, pack, or dispenser together with instructions for administration.

This invention is further illustrated by the following examples which should not be construed as limiting. The contents of all references, patents and published patent applications cited throughout this application are incorporated herein by reference.

### EXAMPLES

### Example 1

### Construction of the SOD1 Fusion Plasmid

SOD1^{G93A}GFP fusion plasmid was constructed as described before (Ding et al. Submitted). Briefly, mutant human SOD1^{G93A} cDNA was PCR cloned between the PmlI and PstI sites of pCMV/myc/mito/GFP (Invitrogen). This cloning step deleted the mitochondrial targeting sequence. U6G93Ahp was constructed as described (6). Similarly, U6misG93A was created using the sequence GACAAAGCTGCTGTATCGGCT (sense strand) (SEQ ID NO: 4), which contains five mismatched nucleotides (bold and underlined) against the SOD1^{G93A} mutant. CMV enhancer was PCR cloned from pDsRed2-N1 vector (1 to 484 nucleotides; Clontech) and inserted either upstream between KpnI and NheI or downstream between NotI and SacI of the U6G93Ahp.

### Example 2

### Cell Culture and Transfection

Human embryonic kidney cell line 293 was grown in DMEM supplemented with 10% fetal bovine serum (FBS), 100 units/ml penicillin, and 100 μg/ml streptomycin. Twenty-four hours before transfection, cells (70-90% confluency) were detached by trituration and transferred to 6-well plates, and cultured in 10% FBS-containing medium without antibiotics. The cells were transfected with 4 μg of the target vector SOD1^{G93A}GFP and 8 μg of each of the hairpin vectors using lipofectamine 2000 (Invitrogen) according to manufacturer's instructions. The transfection efficiency is ˜95% in all experiments. After 24 hrs, the culture medium was changed to DMEM supplemented with 10% FBS and antibiotics. At 40 hrs after transfection, the cells were harvested and quickly frozen in liquid nitrogen.

### Example 3

### Measurement of GFP Fluorescence Intensity

The harvested cells were lysed in ice-cold reporter lysis buffer (Promega) containing protease inhibitors (complete, EDTA-free, 1 tablet/10 ml buffer; Roche Molecular Biochemicals). The lysate was cleared by centrifugation at 16000 RCF and 4° C. for 10 min. The total protein in the cleared lysate was measured using BCA assay (Pierce; Rockville Ill.). Concentration of total protein in each sample was adjusted to 0.5 mg/ml with the reporter buffer. Fluorescence of GFP in 140 μl of samples was measured by fluorescence spectroscopy (Photon Technology International) with excitation at 460 nm and recording from 480 to 600 nm. The spectrum peak was detected at 502 nm, representing the fluorescence intensity of GFP. Fluorescence in the untransfected lysate was measured as background and subtracted from measurements of the transfected lysates.

### Example 4

### Analysis of Western and Northern Blots

Western blot

Twenty micrograms of total proteins were resolved on a 12% SDS-PAGE gel and transferred onto GeneScreen Plus membrane (Perkin Elmer). The membrane was incubated sequentially with a sheep anti-SOD1 (BioDesign) and HRP-labeled goat anti-sheep IgG (Amersham). The protein bands were visualized using SuperSignal kit (Pierce) and Kodak Digital Image Station 440CF.

Northern blot

Cellular RNA was isolated with TRI reagent (Sigma). Twenty μg of total RNA was fractionated on a 15% polyacrylamide gel and transferred to HybondTM-N-+ membrane (Amersham). The membrane was probed with ³²P-labeled synthetic RNA oligonucleotide complementary to the antisense strand of the G93A hairpin.

### Example 5

### Selective Inhibition of Expression of SOD1^{G93A}

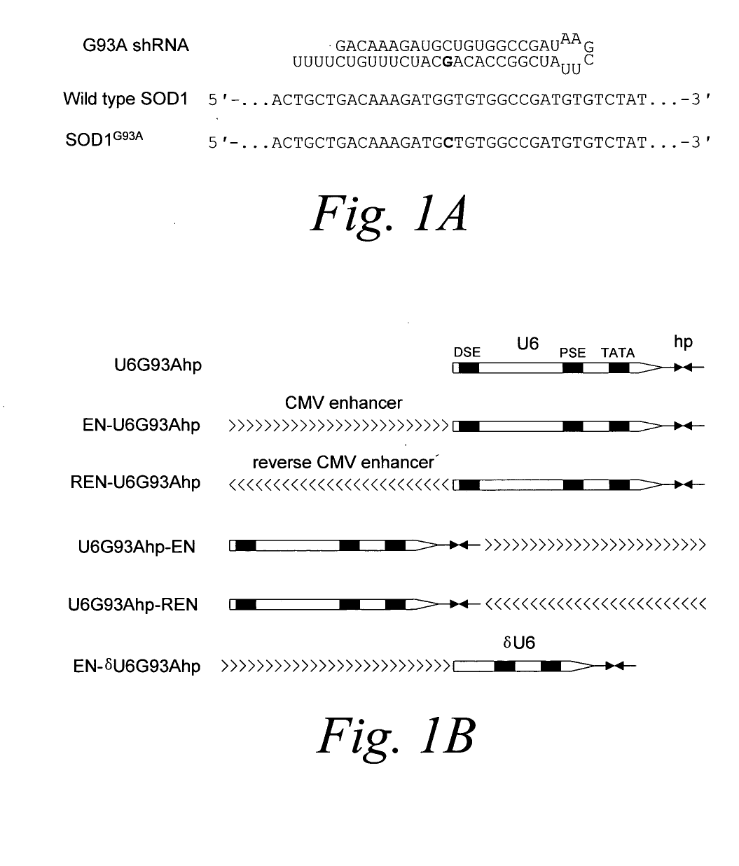

The shRNA against SOD1^{G93A} (G93Ahp) contains a stem that is homologous to SOD1^{G93A} mRNA but has a mismatched nucleotide with SOD1^{WT} at the middle of the stem (FIG. 1A) (SEQ ID NO: 1). In FIG. 1A, the bolded G in the antisense strand of the hairpin is complementary to the bolded C in the SOD1^{G93A} (SEQ ID NO: 3) but forms a mismatch with a G in the SOD1^{WT} (SEQ ID NO: 2).

When transfected into cultured cells, this shRNA selectively inhibited the expression of SOD1^{G93A} but did not affect the expression of SOD1^{WT} (Ding et al. submitted; also see below). The potency of this shRNA was increased by increasing its expression. The U6 promoter was modified by placing the enhancer from the cytomegalovirus (CMV) immediate-early promoter near the U6 promoter, either upstream or downstream from U6G93Ahp and in either forward or backward orientation (FIG. 1B).

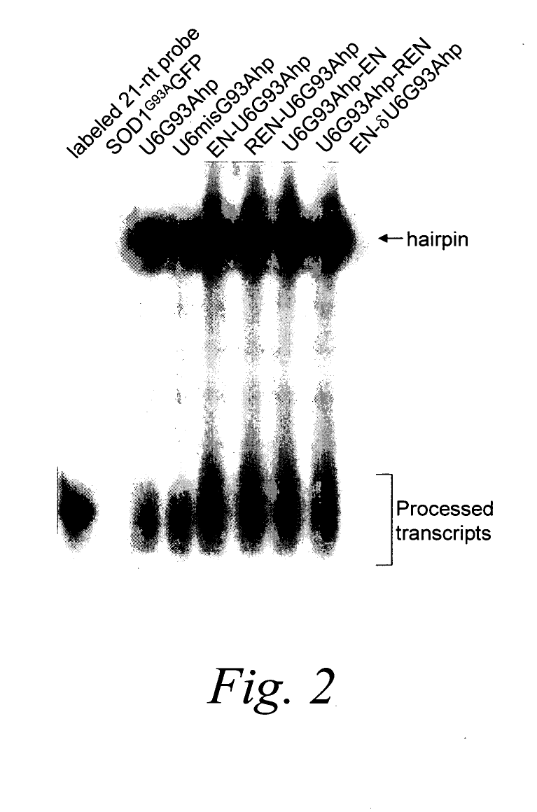

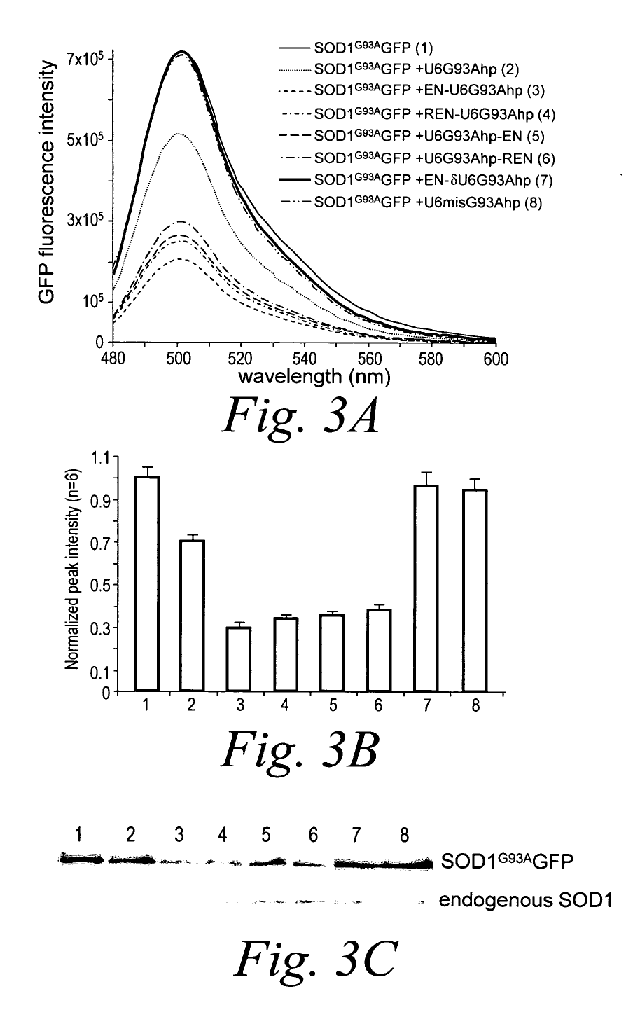

Each of the seven constructs were cotransfectd containing various combinations of U6 promoter, G93Ahp and CMV enhancer, with a target construct that encode a SOD1^{G93A} and GFP fusion protein (SOD1^{G93A}GFP), into the human 293 cells. Northern blot analysis demonstrated that addition of the CMV enhancer near U6G93Ahp in all four configurations (FIG. 1) increased the expression of G93Ahp (FIG. 2). Total RNA was extracted from human 293 cells cotransfected with SOD1^{G93A}GFP and various U6G93Ahp constructs (FIG. 1B). G93Ahp was detected using ³²P-labeled 21-nt RNA probe complementary to the anti-sense strand of the hairpin stem. The enhanced expression of the shRNA in cells transfected with the U6G93Ahp constructs containing the CMV enhancer is evident in FIG. 2. Deletion of the distal sequence element (DSE), an obligatory component of the U6 promoter (25-28), abolished the expression of G93Ahp even in the presence of the enhancer (FIG. 2). Quantification of SOD1^{G93A}GFP expression by the GFP fluorescence using a fluorometer (31) showed that, compared with SOD1^{G93A}GFP alone transfection (#1, FIGS. 3A-3B), G93Ahp produced by the unmodified U6 promoter (U6G93Ahp) inhibited SOD1^{G93A}GFP expression modestly (#2, FIGS. 3A-3B). Attaching the CMV enhancer in all four configurations to the U6G93Ahp (FIG. 1B) enhanced the inhibition of SOD1^{G93A}GFP expression to the similar degree (#3-6, FIGS. 3A-3B). Deletion of the DSE abolished the inhibition of SOD1^{G93A}GFP expression (#7, FIGS. 3A-3B). Finally, U6 promoter directed synthesis of mismatched shRNA did not show any inhibitory activity towards the target gene (#8, FIGS. 3A-3B).

Western blot using a polyclonal anti-SOD1 antibody confirmed the above finding, and furthermore, showed that the enhanced synthesis of G93Ahp only inhibited SOD1^{G93A}GFP expression but did not affect the endogenous human SOD1 levels (FIG. 3c), indicating that the high levels of G93Ahp expression do not affect the specificity of G93Ahp for the mutant SOD1^{G93A}.

The results demonstrate that the CMV enhancer can enhance U6 promoter activity and increase the production of shRNA. This modified promoter may be useful where limited choices of shRNA sequences preclude the selection of highly efficient RNAi target region, and therefore, could be used for selective inhibition of mutant gene expression in vitro and in vivo and developing therapy for diseases caused by dominant, gain-of-function type of gene mutations.

### Example 6

### Transgenic Animals that Express shRNAs

Example 6 teaches how to make and characterize transgenic mice using the compositions and methods of the present invention. The transgenic mice express shRNAs against SOD1^{G93A} and SOD1^{G85R} under the control of a RNA polymerase III (Pol III) promoter U6 (U6-G93A and U6-G85R mice). These mice were crossed with mutant SOD1^{G93A} and SOD1^{G85R} mice, as well as the wild type SOD1^{WT} mice.

A construct has been constructed and tested that qualifies as an effective transgene: the expression of this shRNA mediates efficient and selective inhibition of mutant SOD1^{G93A} expression in culture and in vivo. The transgene was linearized by digestion using Kpn I and Sac I (FIG. 5A), purified and injected into fertilized mouse eggs at University of Massachusetts Medical School (UMMS) transgenic core. To screen for U6-G93A transgenic mice, PCR primers that selectively amplify the transgene sequence were designed and used to identify the transgenic mice. A total of seven founders (F0) were identified and are crossed with FVB, a genetic background that is used to host the SOD1^{G93A} and SOD1^{G85R} mutants.

F1 mice may be analyzed for transgene copy numbers using Southern blot as described previously (Xu et al., 1993). Tail DNA is digested with Bam H1, which will generate a transgene fragment of 388 nucleotides. Because the endogenous mouse U6 promoter has only one BamHI site, the BamHI digestion will produce a larger fragment from the endogenous mouse U6 gene. The ³²P-labeled RNA oligonucleotide probes complementary to the U6 promoter region are used for hybridization. RNA probes generated excellent linear and quantitative Southern and Northern blots. The U6 region is used as target because it is possible to detect the endogenous mouse U6 band together with the transgene on the same blot. Therefore, the endogenous band can be used as the reference for quantifying the transgene copy number.

The expression of shRNA is determined in each line in different tissues, including fore brain, cerebellum, brain stem, spinal cord, heart, liver, kidney and skeletal muscle, using Northern blot.

Transgenic lines expressing different levels of shRNA are crossed with the high as well as the low expressers of SOD1^{G93A}. The high expresser develops weakness at ˜80 days, paralysis at ˜120 days and die at ˜140 days. The low expresser develops weakness at ˜160 days, paralysis at ˜240 days and die at ˜260 days. After breeding for five or more generations, complete analysis on disease phenotype is conducted using mice singly transgenic for SOD1^{G93A} and doubly transgenic for U6-G93A and SOD1^{G93A} (U6-G93A/SOD1^{G93A}).

The levels of SOD1^{G93A} expression will be assessed at the message level by Northern blot and at the protein level by Western blot.

To determine the in vivo specificity of the shRNA, U6-G93A mice are crossed with SOD1^{WT} and another mutant, SOD1^{G85R}, transgenic lines. Levels of SOD1 in different tissues from doubly and singly transgenic mice are measured and compared. The disease progression and survival is also compared between U6-G93A/SOD1^{G85R} doubly transgenic mice and SOD1^{G85R} singly transgenic mice to determine whether U6-G93A has an impact on the disease progression in SOD1^{G85R} mice. If the U6-G93A inhibits the SOD1^{G93A} mutant exclusively, it will not affect the onset and progression of the disease in SOD1^{G85R} mice.

The following references are incorporated herein by reference:
1. Elbashir, S. M., Harborth, J., Lendeckel, W., Yalcin, A., Weber, K. and Tuschl, T. (2001) Duplexes of 21-nucleotide RNAs mediate RNA interference in cultured mammalian cells. Nature, 411, 494-498.
2. Caplen, N. J., Parrish, S., Imani, F., Fire, A. and Morgan, R. A. (2001) Specific inhibition of gene expression by small double-stranded RNAs in invertebrate and vertebrate systems. Proc Natl Acad Sci USA, 98, 9742-9747.
3. Paul, C. P., Good, P. D., Winer, I. and Engelke, D. R. (2002) Effective expression of small interfering RNA in human cells. Nat Biotechnol, 20, 505-508.
4. Lee, N. S., Dohjima, T., Bauer, G., Li, H., Li, M. J., Ehsani, A., Salvaterra, P. and Rossi, J. (2002) Expression of small interfering RNAs targeted against HIV-1 rev transcripts in human cells. Nat Biotechnol, 20, 500-505.
5. Paddison, P. J., Caudy, A. A., Bernstein, E., Hannon, G. J. and Conklin, D. S. (2002) Short hairpin RNAs (shRNAs) induce sequence-specific silencing in mammalian cells. Genes Dev, 16, 948-958.
6. Sui, G., Soohoo, C., Affar el, B., Gay, F., Forrester, W. C. and Shi, Y. (2002) A DNA vector-based RNAi technology to suppress gene expression in mammalian cells. Proc Natl Acad Sci USA, 99, 5515-5520.
7. Yu, J. Y., DeRuiter, S. L. and Turner, D. L. (2002) RNA interference by expression of short-interfering RNAs and hairpin RNAs in mammalian cells. Proc Natl Acad Sci USA, 99, 6047-6052.
8. McManus, M. T., Petersen, C. P., Haines, B. B., Chen, J. and Sharp, P. A. (2002) Gene silencing using micro-RNA designed hairpins. RNA, 8, 842-850.
9. Brummelkamp, T. R., Bernards, R. and Agami, R. (2002) A system for stable expression of short interfering RNAs in mammalian cells. Science, 296, 550-553.
10. Miyagishi, M. and Taira, K. (2002) U6 promoter-driven siRNAs with four uridine 3′ overhangs efficiently suppress targeted gene expression in mammalian cells. Nat Biotechnol, 20, 497-500.
11. Jacque, J. M., Triques, K. and Stevenson, M. (2002) Modulation of HIV-1 replication by RNA interference. Nature, 418, 435-438.
12. Brummelkamp, T., Bernards, R. and Agami, R. (2002) Stable suppression of tumorigenicity by virus-mediated RNA interference. Cancer Cell, 2, 243.
13. Hasuwa, H., Kaseda, K., Einarsdottir, T. and Okabe, M. (2002) Small interfering RNA and gene silencing in transgenic mice and rats. FEBS Lett, 532, 227-230.
14. Xia, H., Mao, Q., Paulson, H. L. and Davidson, B. L. (2002) siRNA-mediated gene silencing in vitro and in vivo. Nat Biotechnol, 20, 1006-1010.
15. Tiscornia, G., Singer, O., Ikawa, M. and Verma, I. M. (2003) A general method for gene knockdown in mice by using lentiviral vectors expressing small interfering RNA. Proc Natl Acad Sci USA, 100, 1844-1848.
16. Rubinson, D. A., Dillon, C. P., Kwiatkowski, A. V., Sievers, C., Yang, L., Kopinja, J., Zhang, M., McManus, M. T., Gertler, F. B., Scott, M. L. et al. (2003) A lentivirus-based system to functionally silence genes in primary mammalian cells, stem cells and transgenic mice by RNA interference. Nat Genet, 33, 401-406.
17. Boutla, A., Delidakis, C., Livadaras, I., Tsagris, M. and Tabler, M. (2001) Short 5′-phosphorylated double-stranded RNAs induce RNA interference in Drosophila. Curr Biol, 11, 1776-1780.
18. Holen, T., Amarzguioui, M., Wiiger, M. T., Babaie, E. and Prydz, H. (2002) Positional effects of short interfering RNAs targeting the human coagulation trigger Tissue Factor. Nucleic Acids Res, 30, 1757-1766.
19. Zeng, Y. and Cullen, B. R. (2003) Sequence requirements for micro RNA processing and function in human cells. Rna, 9, 112-123.
20. Elbashir, S. M., Martinez, J., Patkaniowska, A., Lendeckel, W. and Tuschl, T. (2001) Functional anatomy of siRNAs for mediating efficient RNAi in Drosophila melanogaster embryo lysate. Embo J, 20, 6877-6888.
21. Elbashir, S. M., Harborth, J., Weber, K. and Tuschl, T. (2002) Analysis of gene function in somatic mammalian cells using small interfering RNAs. Methods, 26, 199-213.
22. Kunkel, G. R., Maser, R. L., Calvet, J. P. and Pederson, T. (1986) U6 small nuclear RNA is transcribed by RNA polymerase III. Proc Natl Acad Sci USA, 83, 8575-8579.
23. Cleveland, D. W. and Rothstein, J. D. (2001) From Charcot to Lou Gehrig: deciphering selective motor neuron death in ALS. Nat Rev Neurosci, 2, 806-819.
24. Ding, H. L., Schwarz, D. S., Keene, A., Affar, E. B., Fenton, L., Xia, X. G., Shi, Y., Zamore, P. D. and Xu, Z. S. (2003) Selective silencing by RNAi of a dominant allele that causes amyotrophic lateral sclerosis. Aging Cell, in press.
25. Bark, C., Weller, P., Zabielski, J., Janson, L. and Pettersson, U. (1987) A distant enhancer element is required for polymerase III transcription of a U6 RNA gene. Nature, 328, 356-359.
26. Carbon, P., Murgo, S., Ebel, J. P., Krol, A., Tebb, G. and Mattaj, L. W. (1987) A common octamer motif binding protein is involved in the transcription of U6 snRNA by RNA polymerase III and U2 snRNA by RNA polymerase II. Cell, 51, 71-79.
27. Kunkel, G. R. and Pederson, T. (1988) Upstream elements required for efficient transcription of a human U6 RNA gene resemble those of U1 and U2 genes even though a different polymerase is used. Genes Dev, 2, 196-204.
28. Das, G., Henning, D., Wright, D. and Reddy, R. (1988) Upstream regulatory elements are necessary and sufficient for transcription of a U6 RNA gene by RNA polymerase III. Embo J, 7, 503-512.
29. Lobo, S. M. and Hernandez, N. (1989) A 7 bp mutation converts a human RNA polymerase II snRNA promoter into an RNA polymerase III promoter. Cell, 58, 55-67.
30. Mattaj, I. W., Dathan, N. A., Parry, H. D., Carbon, P. and Krol, A. (1988) Changing the RNA polymerase specificity of U snRNA gene promoters. Cell, 55, 435-442.
31. Chiu, Y.-L. and Rana, T. M. (2002) RNAi in human cells: basic structural and functional features of small interfering RNA. Molecular Cell, 10, 549-561.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A construct comprising a nucleotide sequence encoding a shRNA operably linked to a Pol III promoter and a Pol II enhancer.

2. The construct of claim 1, wherein the Pol III promoter is selected from a group consisting of a U6 promoter, a H1 promoter, and a tRNA promoter.

3. The construct of claim 1, wherein the Pol III promoter is a U6 promoter.

4. The construct of claim 1, wherein the Pol II enhancer is a CMV enhancer.

5. A construct comprising a nucleotide sequence encoding a shRNA operably linked to a Pol II promoter and a Pol III enhancer.

6. The construct of claim 5, wherein the Pol II promoter is a CMV promoter.

7. The construct of any one of the proceeding claims, wherein the shRNA comprises a sequence sufficiently complementary to a target mRNA to mediate degradation of said target.

8. The construct of claim 7, wherein said target mRNA encodes a wild type protein.

9. The construct of claim 7, wherein said target mRNA encodes a mutant protein.

10. The construct of claim 9, wherein said mutant protein is a gain-of-function mutant.

11. The construct of claim 9, wherein said mutant protein is a disease-causing mutant.

12. The construct of any one of claims 9-11, wherein said mutant protein is SOD1.

13. The construct of claim 12, wherein said mutant protein is SOD1^{G93A}.

14. The construct of claim 12, wherein said mutant protein is SOD1^{G85R}.

15. The construct of claim 9, wherein said mutant protein is causative of a neurological disease or disorder.

16. The construct of claim 15, wherein said neurological disease or disorder is a neurodegenerative disease or disorder.

17. The construct of claim 16, wherein said neurodegenerative disease is selected from the group consisting of a neurodegenerative disease, Lou Gehrig's disease, amyotrophic lateral sclerosis (ALS), Alzheimer's disease, Parkinson's disease, Adrenoleukodystrophy (ALD), and dementia.

18. A cell comprising a construct of any one of claims 1-17.

19. A vector comprising the construct of any one of claims 1-17.

20. The vector of claim 19, wherein said vector is a viral vector.

21. The vector of claim 19, wherein said vector is selected from the group consisting of AAV, Lentiviral, Adenoviral and Herpes vectors.

22. A cell comprising the vector of any one of claims 19-21.

23. The cell of claim 18 or 22, wherein the cell is an animal cell.

24. The construct of any one of claims 1-17, wherein the enhancer is upstream from the promoter.

25. The construct of any one of claims 1-17, wherein the enhancer is downstream from the promoter.

26. The construct of any one of claims 1-17, wherein the enhancer in a forward orientation.

27. The construct of any one of claims 1-17, wherein the enhancer in a backward orientation.

28. A composition comprising the construct of any one of claims 1-17 and a pharmaceutically acceptable carrier.

29. A construct comprising a nucleotide sequence selected from SEQ ID NOS:1-7.

30. A nonhuman transgenic animal carrying a transgene comprising the constructs of any one of claims 1-17, 24-27 and 29.

31. A nonhuman homologous recombinant animal which contains the cell of any one of claims 18, 22 and 23.

32. A method for enhancing RNAi, the method comprising introducing into a cell the construct of any one of claims 1-17, 24-27 and 29 under conditions such that shRNA expression is increased, thereby enhancing RNAi.

33. The method of claim 32, wherein the cell is present in a subject.

34. The method of claim 32, wherein the cell is a cultured cell.

35. The method of claim 32, wherein said introducing comprises transfecting said cell.

36. The method of claim 32, wherein said introducing comprises infecting said cell with a viral vector.

37. A method of enhancing RNAi in a subject, the method comprising administering the construct of any one of claims 1-17, 24-27 and 29 or the composition of any one of claims 24-28, thereby enhancing RNAi in a subject.

38. A method for selectively inhibiting mutant gene expression in vivo or in vitro, the method comprising introducing into a host cell the construct of any one of claims 1-17 and 29 under conditions such that said shRNA is expressed, thereby inhibiting mutant gene expression.

39. The method of claim 38, wherein the shRNA does not inhibit expression of the wild type allele.

40. A method for treating a disease in a subject, the method comprising administering the construct of any one of claims 1-17, 24-27 and 29 or the composition of any one of claims 24-28, thereby treating a disease in a subject.

41. The method of claim 40, wherein the disease is selected from the group consisting of a neurodegenerative disease, Lou Gehrig's disease, amyotrophic lateral sclerosis (ALS), Alzheimer's disease, Parkinson's disease, Adrenoleukodystrophy (ALD), and dementia.

42. The method of claim 40, wherein the disease is caused by a mutation that is a dominant, gain-of-function mutation.

43. A method for identifying a compound which modulates RNAi, the method comprising:
(a) contacting a cell comprising the construct of any one of claims 1-17, 24-27 and 29 with a test compound; and
(b) determining the effect of the test compound on an indicator of RNAi activity in said cell, thereby identifying a compound which modulates RNAi.

44. A compound identified according to the method of claim 43.

45. A method for modulating RNAi, the method comprising contacting a cell expressing the construct of any one of claims 1-17, 24-27 and 29 with the compound of claim 44 in a sufficient concentration to modulate the activity of RNAi.

46. A method for deriving information about the function of a gene in a cell or organism comprising:
(a) introducing into said cell or organism the construct of any one of claims 1-17, 24-27 and 29;
(b) maintaining the cell or organism under conditions such that RNAi can occur;
(c) determining a characteristic or property of said cell or organism; and
(d) comparing said characteristic or property to a suitable control,
the comparison yielding information about the function of the gene.

47. A method of validating a candidate protein as a suitable target for drug discovery comprising:
(a) introducing into a cell or organism the construct of any one of claims 1-17, 24-27 and 29;
(b) maintaining the cell or organism under conditions such that RNAi can occur;
(c) determining a characteristic or property of said cell or organism; and
(d) comparing said characteristic or property to a suitable control,
the comparison yielding information about whether the candidate protein is a suitable target for drug discovery.

48. A kit comprising reagents for activating RNAi in a cell or organism, said kit comprising:
(a) the construct of any one of claims 1-17, 24-27 and 29; and
(b) instructions for use.


SEQUENCE LISTING (4 sequences)

SEQ ID NO: 1 - DNA, 52 bp, Artificial Sequence
  synthetic ShRNA
gacaaagaugcuguggccgauaagcuuaucggccacagcaucuuugucuuuu

SEQ ID NO: 2 - DNA, 35 bp, Artificial Sequence
  synthetic SiRNA target sequence
actgctgacaaagatggtgtggccgatgtgtctat

SEQ ID NO: 3 - DNA, 35 bp, Artificial Sequence
  synthetic SiRNA target sequence
actgctgacaaagatgctgtggccgatgtgtctat

SEQ ID NO: 4 - DNA, 21 bp, Artificial Sequence
  synthetic construct
gacaaagctgctgtatcggct